SEQUENCE LISTING (13 sequences)

SEQ ID NO: 1 - protein, 25 aa, Homo sapiens
TLDPRSFLLRNPNDKYEPFWEDEEK

SEQ ID NO: 2 - protein, 25 aa, Homo sapiens
SSKGRSLIGKVDGTSHVTGKGVTVE

SEQ ID NO: 3 - protein, 24 aa, Homo sapiens
TLPIKTFRGAPPNSFEEFPFSALE

SEQ ID NO: 4 - protein, 25 aa, Homo sapiens
LPAPRGYPGQVCANDSDTHELPDSS

SEQ ID NO: 5 - DNA, 3480 bp, Homo sapiens
  CDS (225)..(1502)
gcgcccgcgcgaccgcgcgccccagtcccgccccgccccgctaaccgccccagacacagcgctcgccgagggtcgcttggaccctgatcttacccgtgggcaccctgcgctctgcctgccgcgaagaccggctccccgacccgcagaagtcaggagagagggtgaagcggagcagcccgaggcggggcagcctcccggagcagcgccgcgcagagcccgggacaatggggccgcggcggctgctgctggtggccgcctgcttcagtctgtgcggcccgctgttgtctgcccgcacccgggcccgcaggccagaatcaaaagcaacaaatgccaccttagatccccggtcatttcttctcaggaaccccaatgataaatatgaaccattttgggaggatgaggagaaaaatgaaagtgggttaactgaatacagattagtctccatcaataaaagcagtcctcttcaaaaacaacttcctgcattcatctcagaagatgcctccggatatttgaccagctcctggctgacactctttgtcccatctgtgtacaccggagtgtttgtagtcagcctcccactaaacatcatggccatcgttgtgttcatcctgaaaatgaaggtcaagaagccggcggtggtgtacatgctgcacctggccacggcagatgtgctgtttgtgtctgtgctcccctttaagatcagctattacttttccggcagtgattggcagtttgggtctgaattgtgtcgcttcgtcactgcagcattttactgtaacatgtacgcctctatcttgctcatgacagtcataagcattgaccggtttctggctgtggtgtatcccatgcagtccctctcctggcgtactctgggaagggcttccttcacttgtctggccatctgggctttggccatcgcaggggtagtgcctctcgtcctcaaggagcaaaccatccaggtgcccgggctcaacatcactacctgtcatgatgtgctcaatgaaaccctgctcgaaggctactatgcctactacttctcagccttctctgctgtcttcttttttgtgccgctgatcatttccacggtctgttatgtgtctatcattcgatgtcttagctcttccgcagttgccaaccgcagcaagaagtcccgggctttgttcctgtcagctgctgttttctgcatcttcatcatttgcttcggacccacaaacgtcctcctgattgcgcattactcattcctttctcacacttccaccacagaggctgcctactttgcctacctcctctgtgtctgtgtcagcagcataagctcgtgcatcgaccccctaatttactattacgcttcctctgagtgccagaggtacgtctacagtatcttatgctgcaaagaaagttccgatcccagcagttataacagcagtgggcagttgatggcaagtaaaatggatacctgctctagtaacctgaataacagcatatacaaaaagctgttaacttaggaaaagggactgctgggaggttaaaaagaaaagtttataaaagtgaataacctgaggattctattagtccccacccaaactttattgattcacctcctaaaacaacagatgtacgacttgcatacctgctttttatgggagctgtcaagcatgtatttttgtcaattaccagaaagataacaggacgagatgacggtgttattccaagggaatattgccaatgctacagtaataaatgaatgtcacttctggatatagctaggtgacatatacatacttacatgtgtgtatatgtagatgtatgcacacacatatattatttgcagtgcagtatagaataggcactttaaaacactctttccccgcaccccagcaattatgaaaataatctctgattccctgatttaatatgcaaagtctaggttggtagagtttagccctgaacatttcatggtgttcatcaacagtgagagactccatagtttgggcttgtaccacttttgcaaataagtgtattttgaaattgtttgacggcaaggtttaagttattaagaggtaagacttagtactatctgtgcgtagaagttctagtgttttcaattttaaacatatccaagtttgaattcctaaaattatggaaacagatgaaaagcctctgttttgatatgggtagtattttttacattttacacactgtacacataagccaaaactgagcataagtcctctagtgaatgtaggctggctttcagagtaggctattcctgagagctgcatgtgtccgcccccgatggaggactccaggcagcagacacatgccagggccatgtcagacacagattggccagaaaccttcctgctgagcctcacagcagtgagactggggccactacatttgctccatcctcctgggattggctgtgaactgatcatgtttatgagaaactggcaaagcagaatgtgatatcctaggaggtaatgaccatgaaagacttctctacccatcttaaaaacaacgaaagaaggcatggacttctggatgcccatccactgggtgtaaacacatctagtagttgttctgaaatgtcagttctgatatggaagcacccattatgcgctgtggccactccaataggtgctgagtgtacagagtggaataagacagagacctgccctcaagagcaaagtagatcatgcatagagtgtgatgtatgtgtaataaatatgtttcacacaaacaaggcctgtcagctaaagaagtttgaacatttgggttactatttcttgtggttataacttaatgaaaacaatgcagtacaggacatatattttttaaaataagtctgatttaattgggcactatttatttacaaatgttttgctcaatagattgctcaaatcaggttttcttttaagaatcaatcatgtcagtctgcttagaaataacagaagaaaatagaattgacattgaaatctaggaaaattattctataatttccatttacttaagacttaatgagactttaaaagcattttttaacctcctaagtatcaagtatagaaaatcttcatggaattcacaaagtaatttggaaattaggttgaaacatatctcttatcttacgaaaaaatggtagcattttaaacaaaatagaaagttgcaaggcaaatgtttatttaaaagagcaggccaggcgcggtggctcacgcctgtaatcccagcactttgggaggctgaggcgggtggatcacgaggtcaggagatcgagaccatcctggctaacacggtgaaacccgtctctactaaaaatgcaaaaaaaattagccgggcgtggtggcaggcacctgtagtcccagctactcgggaggctgaggcaggagactggcgtgaacccaggaggcggaccttgtagtgagccgagatcgcgccactgtgctccagcctgggcaacagagcaagactccatctcaaaaaaaa

SEQ ID NO: 6 - protein, 425 aa, Homo sapiens
MGPRRLLLVAACFSLCGPLLSARTRARRPESKATNATLDPRSFLLRNPNDKYEPFWEDEEKNESGLTEYRLVSINKSSPLQKQLPAFISEDASGYLTSSWLTLFVPSVYTGVFVVSLPLNIMAIVVFILKMKVKKPAVVYMLHLATADVLFVSVLPFKISYYFSGSDWQFGSELCRFVTAAFYCNMYASILLMTVISIDRFLAVVYPMQSLSWRTLGRASFTCLAIWALAIAGVVPLVLKEQTIQVPGLNITTCHDVLNETLLEGYYAYYFSAFSAVFFFVPLIISTVCYVSIIRCLSSSAVANRSKKSRALFLSAAVFCIFIICFGPTNVLLIAHYSFLSHTSTTEAAYFAYLLCVCVSSISSCIDPLIYYYASSECQRYVYSILCCKESSDPSSYNSSGQLMASKMDTCSSNLNNSIYKKLLT

SEQ ID NO: 7 - DNA, 548 bp, Homo sapiens
cgccgagggtcgcttggaccctgatcttacccgtgggcaccctgcgctctgcctgccgcgaagaccggctccccgacccgcagaagtcaggagagagggtgaagcggagcagcccgaggcggggcagcctcccggagcagcgccgcgcagagcccgggacaatggggccgcggcggctgctgctggtggccgcctgcttcagtctgtgcggcccgctgttgtctgcccgcacccgggcccgcaggccagaatcaaaagcaacaaatgccaccttagatccccggtcatttcttctcaggaaccccaatgataaatatgaaccattttgggaggatgaggagaaaaatgaaagtgggttaactgaatacagattagtctccatcaataaaagcagtcctcttcaaaaacaacttcctgcattcatctcagaagatgcctccggatatttgaccagctcctggctgacactctttgtcccatctgtgtacaccggagtgtttgtagtcagcctcccactaaacatcatggccatcgttgtgttcatcctg

SEQ ID NO: 8 - DNA, 1289 bp, Homo sapiens
aaaatgaataaatgaatgtactttcatttgaacaaaccagtgttactgctgaaacatttatttctgtaatgacccttgtcttcctttcttgtacaggaaccaatagatcctctaaaggaagaagccttattggtaaggttgatggcacatcccacgtcactggaaaaggagttacagttgaaacagtcttttctgtggatgagttttctgcatctgtcctcactggaaaactgaccactgtcttccttccaattgtctacacaattgtgtttgtggtgggtttgccaagtaacggcatggccctgtgggtctttcttttccgaactaagaagaagcaccctgctgtgatttacatggccaatctggcgttggcggacctcctctctgtcatctggttccccttgaagattgcctatcacatacatggcaacaactggatttatggggaagctctttgtaatgtgcttattggctttttctatggtaacatgtactgttccattctcttcatgacctgcctcagtgtgcagaggtattgggtcatcgtgaaccccatggggcactccaggaagaaggcaaacattgccattggcatctccctggcaatatggctgctgattcttctggtcaccatccctttgtatgtcgtgaagcagaccatcttcattcctgccctgaacatcacgacctgccatgatgttttgcctgagcagctcttggtgggagacatgttcaattacttcctctctctggccattggggtctttctgttcccagccttcctcacagcctctgcctatgtgctgatgatcagaatgctgcgatcttctgccatggatgaaaactcagagaagaaaaggaagagggccatcaaactcattgtcactgtcctggccatgtacctgatctgcttcactcctagtaaccttctgcttgtggtgcattattttctgattaaaagccagggccagagccatgtctatgccctgtacattgtagccctctgcctctctacccttaacagctgcatcgacccctttgtctattactttgtttcacatgatttcagggatcatgcaaagaacgctctcctttgccgaagtgtccgcactgtaaagcagatgcaagtatccctcacctcaaagaaacactccaggaaatccagctcttactcttcaagttcaaccactgttaagacctcctattgagttttccaggtcctcagatgggaattgcacagtaggatgtggaacctgtttaatgttatgaggacgtgtctgttatttcct

SEQ ID NO: 9 - DNA, 1830 bp, Homo sapiens
cctgcctgcacggcacaggagagcaaacttctacagacagaccaaggcttccatttgctgctgacacatggaactgaggtgaaattgtgctccatgattttacagatttcataacgtttaagagacgggactcaggtcatcaaaatgaaagccctcatctttgcagctgctggcctcctgcttctgttgcccactttttgtcagagtggcatggaaaatgatacaaacaacttggcaaagccaaccttacccattaagacctttcgtggagctcccccaaattcttttgaagagttccccttttctgccttggaaggctggacaggagccacgattactgtaaaaattaagtgccctgaagaaagtgcttcacatctccatgtgaaaaatgctaccatggggtacctgaccagctccttaagtactaaactgatacctgccatctacctcctggtgtttgtagttggtgtcccggccaatgctgtgaccctgtggatgcttttcttcaggaccagatccatctgtaccactgtattctacaccaacctggccattgcagattttcttttttgtgttacattgccctttaagatagcttatcatctcaatgggaacaactgggtatttggagaggtcctgtgccgggccaccacagtcatcttctatggcaacatgtactgctccattctgctccttgcctgcatcagcatcaaccgctacctggccatcgtccatcctttcacctaccggggcctgcccaagcacacctatgccttggtaacatgtggactggtgtgggcaacagttttcttatatatgctgccatttttcatactgaagcaggaatattatcttgttcagccagacatcaccacctgccatgatgttcacaacacttgcgagtcctcatctcccttccaactctattacttcatctccttggcattctttggattcttaattccatttgtgcttatcatctactgctatgcagccatcatccggacacttaatgcatacgatcatagatggttgtggtatgttaaggcgagtctcctcatccttgtgatttttaccatttgctttgctccaagcaatattattcttattattcaccatgctaactactactacaacaacactgatggcttatattttatatatctcatagctttgtgcctgggtagtcttaatagttgcttagatccattcctttattttctcatgtcaaaaaccagaaatcactccactgcttaccttacaaaatagtgaaatgatcttagagaacaaggacagccatcacagagaacgtctgttttcaagaacaacataagcatagtgcaaggagctccatttccgagctcctaagaaatatgcttcaaaggtcaaacattacaaaagcattagtagtttgtttgtttgtttttgagactgagtctcactttatcacccagactggcgtgcagtggcactatcttggctcattgcaacctctgcctcccaggtcagcctcccaagtagctgggattacaccaccatgcccagctactaaaaatacttgtatttttagtagagacggggtttcaccatgttgaccaggctggtcttgaactcctgacctcaagtgatcttccggcctcagcctcccaaagtgctggattacaggcgtgagccactgagccagccagcattagtaatttttaaaaacactttatcagtattttaaaaatgttaatgcaggagaaaagatatcacaactctatggaaaatgacatttccatttgccttattgctacttcaagctctttaaatcaccatcttccctatttc

SEQ ID NO: 10 - DNA, 4895 bp, Homo sapiens
  CDS (176)..(1333)
ctcccacgggctggctggcaagcggccctggtgggtctgcgggggcaggggcagccttcctggtttatctccaccggcgcgatctgctcgtccgcctcggctccagaagctggggctcagggtccggcgaggcaggaagcctgaggccacagcccagagcagcctgagtgcagtcatgtgggggcgactgctcctgtggcccctggtgctggggttcagcctgtctggcggcacccagacccccagcgtctacgacgagagcgggagcaccggaggtggtgatgacagcacgccctcaatcctgcctgccccccgcggctacccaggccaagtctgtgccaatgacagtgacaccctggagctcccggacagctcacgggcactgcttctgggctgggtgcccaccaggctggtgcccgccctctatgggctggtcctggtggtggggctgccggccaatgggctggcgctgtgggtgctggccacgcaggcacctcggctgccctccaccatgctgctgatgaacctcgcgactgctgacctcctgctggccctggcgctgcccccgcggatcgcctaccacctgcgtggccagcgctggcccttcggggaggccgcctgccgcctggccacggccgcactctatggtcacatgtatggctcagtgctgctgctggccgccgtcagcctggatcgctacctggccctggtgcacccgctgcgggcccgcgccctgcgtggccggcgcctggcccttggactctgcatggctgcttggctcatggcggccgccctggcactgcccctgacactgcagcggcagaccttccggctggcgcgctccgatcgcgtgctctgccatgacgcgctgcccctggacgcacaggcctcccactggcaaccggccttcacctgcctggcgctgttgggctgtttcctgcccctgctggccatgctgctgtgctacggggccaccctgcacacgctggcggccagcggccggcgctacggccacgcgctgaggctgaccgcagtggtgctggcctccgccgtggccttcttcgtgcccagcaacctgctgctgctgctgcattactcggacccgagccccagcgcctggggcaacctctatggtgcctacgtgcccagcctggcgctgagcaccctcaacagctgcgtggatcccttcatctactactacgtgtcggccgagttcagggacaaggtgcgggcagggctcttccaacggtcgccgggggacaccgtggcctccaaggcctctgcggaagggggcagccggggcatgggcacccactcctctttgctccagtgacacaaagtggggaaggctgtactgggtcgaacagggtcccttcccccacttcacgtccttcctgggacctcagaatgtgaccttatttggaaatagggttgttacaactgtcactagcggaggtcactttggagaagggtgggccttacatccagtgtgggtggtgtcctcataagataaggagaggccaggcctggtggctcacgcctgtaatcccagcactttaagaggccaaggcggatggatcacttgagcccaggagttcaacaccagcctgagcaacatggtaaaaccccatctctaccaaaaatacaaaaattagctgggcttggtggctggcgcctgtaatcccagctactcaggagactgaggcagaaggatcgcttgaacctgggaggcagaggttgcagtgagccgagattgcgccactggactccagcctgcgtgacagagagcctgtctctaaattaattaattaattaatttaattcaattttaaaaagacgaaaagtgacggccaggtgcagtggctcacgcctataatctcagcactctgggaggccaagatggaggattgcttgaagccaggagtttgggaccagcctgggcaacatagggggatcccatctctacacacaaaaaaattttttaatgaaccaggcattgtggcatgcgcctatagtcccagccactcaagaggcacaggcgggaggatcacttgagcctgggaggttgtggttgcagtgagctatgattgtaccactgcactccagcctgggcaacagagcaagaccttgtctcaaaaataaacaaactaaaattaaaaaaagaagacgagagatagtgggtgtggtggctcacacctgcaatcccagcactttggaaggccgaggtgggcagatcatctgaggccaggagttcaagaccagcctggctaacatggtgaaatcctatctctaccaaaaatacaaaaattagccaggcgtggtggtgggcacctgtactggggaggtgcccacccagctactggggaggctgagtcaggagaatcgcttgaacctgggaggcggaggttgcggtcagctgagatggtgccactgcactccagcctgggcgaaagagcgactctgtctccaaaaaaaagagaagaggagaggacacagagacacacagagaagaaagccatgtggcggcagaggcagagatgggagtgatgcggacggacacaaactaagggatgccacgatgccaagcacagccaacagccaccagcagccaggagacaggcctgggacgggctctccctcacagcctccagagggaaccagccctgccaccaccttgaccctggacttctggcctgcagaactgtgagacaataaactctcattgttttaagctgcctggcatgtggcactttgtcagggcagcccaggaatctgaaacaggatcaaactctgcttcctgggccctgccagcatctctggctcggctttctgggctggatgcagcccacgacgcactggtgtctgagatggggctggagctggggctggggctgcattccctggagactcactgcaagttcctgcccaggaggctgagggcaccccatcctcagtgcccaatgctgtggccccaccaggcccagagcctggttggccattctcatgcccaccagcttctggctttgggatgtctcttgagcaaccagaatagcacccccaactctgctccccaaaacccatcactagcacggctcagcctcctgctatcccctgactgctggggaccctcgccttccctcctctcacctgcaggctgatccttcttttcactttctgtcaatgtcaccagggataaggtgggacaatggggggtgggggtggacagtgtgtgctggggggttcgggtgctgcagacctggaactcccttctgccaggatgttggcagccggttgtaagccttgcacgggacagaccacacccaccgcaacctcatcccctcagcactaaccacatccactctcaaccccgtccccttcgcactgaccacacccaccccgttcggccccgccccccgcactgaacactcccgccctcaaccccgcaccctccgcactcacctccccctcgccgctcgaccccgccctcaccacactgaccaccctcaacccattgcgcccagtccccaccacagtgaccacaccctcactggctcggccctgcccccagtatactgaccattccccagccacttcccttccgcacttaccactcccccagccacgcccctccccgctgaccgctcctccagccccgcctcccccgtacaggcagagcgcccgcccacctctatgctgcgttctcctgactttacgttggcccctcctctgccaagcccccaggggagccctccctggcgtccgagggtgggagtcggggtgtggcaggccgcggtggggggcggcagtggctccgcgcactcacccgggccccgggcaggggcgcgctccacttcgttgcacgcgggtccggcgcacagttcccgggcgagtgggctgtgcgtgctgacgttgtagaagcgagtggcctcgaaggctacgggacgagggtggcgggtgaccaagtgcaggcgcgacgggtcagggaccgggccgggccgggggtgcgggcgcgcgggcctaccgggttcgtagtagtcgtacacggagactggcagcgccgacgtcctgcccaccacgcactcccggagagcacggaaccgcacgcacgtcaggcaccggctggggatctgtggggcagcggcgggcgcaggctcgacccgggccaggaggcccggggcgctgagctcaggcccagaactggctgatttcagggatacccaggacgcgtgaaacacagaagaaacgtgatcccattttctttttttcttttacttttctttttttttttttttcctgagacagagtctcgcgctgttgcccaggctggagtgcagtggcgtgatctcggctcactgcaagctcggcctcctgggttcaaatgattctcctgcctcagcctcccaagtagctgggataacaggcgcccaccaccgcaccctgctaattttttgtatttttgatcaagacggagtttcaccatgttggccaggctggtctccaactcctgccctcaagtgatccgcctcggtcccattttttattctttgggtccttccatcccactgggaaaacgtctcaggtggcctctgaaacaccactcctttttgtgtgtgtgcacgcatggctgagcatgtgtgggtgggagtcagcacattcacgatactgtgcaatcatcacctctgtctagttacaggacggtttctttctcccccaaagaaaccccatcgccatcagcactcactccccactcccccagcccctggcaaccacaaatctttccaactctacggatttgcctgttctgggcatttcatgtcaatggaatcatgtactctgtgaaaaaaaaaaaaaaaaaaaaaaaaaaaaaaaaaaaaaaaaaaaaaaaaaaaaaaaaa

SEQ ID NO: 11 - protein, 385 aa, Homo sapiens
MWGRLLLWPLVLGFSLSGGTQTPSVYDESGSTGGGDDSTPSILPAPRGYPGQVCANDSDTLELPDSSRALLLGWVPTRLVPALYGLVLVVGLPANGLALWVLATQAPRLPSTMLLMNLATADLLLALALPPRIAYHLRGQRWPFGEAACRLATAALYGHMYGSVLLLAAVSLDRYLALVHPLRARALRGRRLALGLCMAAWLMAAALALPLTLQRQTFRLARSDRVLCHDALPLDAQASHWQPAFTCLALLGCFLPLLAMLLCYGATLHTLAASGRRYGHALRLTAVVLASAVAFFVPSNLLLLLHYSDPSPSAWGNLYGAYVPSLALSTLNSCVDPFIYYYVSAEFRDKVRAGLFQRSPGDTVASKASAEGGSRGMGTHSSLLQ

SEQ ID NO: 12 - protein, 7 aa, Artificial Sequence
  extracellular loop
ITTCHDV

SEQ ID NO: 13 - protein, 10 aa, Artificial Sequence
  thrombin-receptor activating peptide
SFLLRNPNDK

(19) United States
(12) Patent Application Publication
(10) Pub. No.: US 2008/0045474 A1
(43) Pub. Date: Feb. 21, 2008

(54) Method for treatment of invasive cells

(75) Inventor: Rachel Bar-Shavit, Ramat-Sharet (IL)

Correspondence Address: OLIFF & BERRIDGE, PLC, P.O. BOX 320850, ALEXANDRIA, VA, 22320-4850, US

(73) Assignee: Hadasit Medical Research Services & Development Limited, Jerusalem (IL)

(21) Appl. No.: 11/785,721

(22) Filed: Apr. 19, 2007

Related U.S. Application Data
(63) Continuation of application No. 09/744,679, filed on Apr. 11, 2001, now abandoned.

(30) Foreign Application Priority Data
Aug. 7, 1998 (IL) 125698

(51) Int. Cl. A61K 31/7052; A61P 43/00; C07H 21/00; C12N 15/00; C12N 5/08

(52) U.S. Cl. 514/44; 435/320.1; 435/375; 536/23.1

(57) ABSTRACT

A method for treating metastatic tumor cells of a subject is disclosed. The method comprises administrating to the subject an antisense molecule comprising a nucleotide sequence which is complementary to an RNA sequence of a protease activated receptor (PAR) protein, or an antibody molecule capable of binding to a PAR protein. A method is also described for the treatment of disorders involving the implantation of a placenta in a female subject comprising administrating to the subject the antisense molecule. Also disclosed is the antisense molecule and a pharmaceutical composition comprising it.

### FIELD OF THE INVENTION

This invention relates to the therapeutic use of molecules associated with protease activated receptors.

### BACKGROUND OF THE INVENTION

References referred to by bracketed numbers in the body of the specification are listed at the end of the specification before the claims.

The process by which epithelial cells become invasive is complex and has yet to be fully elucidated. One example of this process is observed in metastatic tumors. Another example of epithelial cells becoming invasive occurs during normal human embryonic development, in which the cytotrophoblasts (i.e. the fetal cells on the front line of the placenta) invade the uterus, as part of their normal differentiation program and successful implantation.

The physiologic invasiveness of cytotrophoblasts closely resembles that of malignant cells, sharing many common features. Tumor invasion and metastasis involve, among other alterations, proteolytic modification of basement membranes and extracellular matrices (ECMs). Cancer cells have to detach from their primary location, encounter basement membranes (i.e. during extravasation of blood or lymphatic vessels), and disseminate through the circulation to establish new cellular colonies at distant sites. Therefore, the process of cell invasion involves a well-orchestrated sequence of events including integrin activation, cell migration and proteolytic degradation of specific barrier components. This enzymatic cleavage is highly regulated, since extensive proteolysis could impede the invasive process by degrading essential matrix components required for the transmission of survival and cell shape signals, through contacts with the basement membrane. Localized proteolysis directed to discrete regions of the cell surface may facilitate cellular invasion.

The thrombin-receptor (ThR) is a seven transmembrane domain G-coupled protein, belonging to the protease-activated receptor (PAR) family [1]. Recently, two other members of this family (PAR-2 and PAR-3) have been identified [2-4], and a fourth member (PAR-4) has also been described [19]. Unlike most cellular growth factor receptors, the activation of these receptors does not require formation of the traditional ligand-receptor complex. Instead, the receptor serves as a substrate for proteolytic digestion, yielding an irreversible form of activated cell surface protein to convey further cell signaling.

Applicant's co-pending Israel Patent Application No. 114890, whose contents are incorporated herein by reference, discloses' that a direct correlation exists between ThR level of expression in tumor cells and their degree of invasiveness. This finding was used to develop a diagnostic method for evaluating the metastatic tendency of tumor cells by following the expression of the ThR gene.

U.S. Pat. No. 5,352,664 to Carney, et al, describes thrombin-derived polypeptides which are capable of selectively stimulating or inhibiting thrombin receptor occupancy signals. Carney suggests that the inhibitory polypeptides may be used in preventing metastasis and angiogenesis. No supporting data is disclosed.

### SUMMARY OF THE INVENTION

It is an object of the present invention to provide a method for treating metastatic tumors.

It is a further object of the present invention to provide a method for treating irregularities in physiological placental development.

The present invention is based on the surprising finding that interfering with the expression of PAR proteins of an invasive cell affects its degree of invasiveness. The interference may be realized at the DNA (gene) level, at the mRNA level, and/or at the protein (receptor) level. Interference at the DNA level may be achieved by use of gene therapy methods; interference at the mRNA level may be achieved by use of antisense molecules; and interference at the protein level may be achieved by use of specific antibodies.

The PAR protein may be any member of the PAR family such as, for example but not limited to, ThR, PAR-2, PAR-3 and PAR-4.

In a first aspect of the invention, the invasive cells are pathological cells such as metastatic tumor cells. Thus, in this aspect of the invention, there is provided a method for treating metastatic tumor cells of a subject comprising administrating to said subject an antisense molecule, said antisense molecule comprising a nucleotide sequence which is complementary to an RNA sequence of a PAR protein.

Also provided are antisense molecules and pharmaceutical compositions comprising them.

Further provided is a method for treating metastatic tumor cells of a subject comprising administrating to said subject an antibody molecule, said antibody molecule being capable of binding to a protease activated receptor (PAR) protein. The antibody molecule may be a polyclonal or monoclonal antibody, prepared by methods known per se.

In this aspect of the invention, the tumor cells will generally be of epithelial origin, which form solid carcinoma-type tumors. Examples of such epithelial tissues are breast, esophagus, kidney, prostate, ovary, melanoma and bladder tissue.

In a second aspect of the invention, the invasive cells are normal cells such as placental cells. As described above, ThR plays a role during cytotrophoblast invasion and implantation. The finding that ThR expression is associated with the invasiveness of placental tissue may be beneficial for improved implantation of human embryo in the maternal uterus decidua. To date, the rate of spontaneous abortions is 8-12%, 50% of which are due to defects in proper implantation. It is even more striking in the I.V.F. procedure, where 40% of the overall cases result in failure. 90% of these failures are apparently due to implantation defects. Transfection of normal placenta with ThR and other PAR family genes may considerably improve implantation.

Thus, in this aspect of the invention, there is provided a method for the treatment of disorders involving the implantation of a placenta in a female subject comprising administrating to said subject an antisense molecule, said antisense molecule comprising a nucleotide sequence which is complementary to an RNA sequence of a PAR protein.

Also provided are antisense molecules and pharmaceutical compositions comprising them.

The synthesis of antisense molecules to known mRNA sequences is well known to the skilled artisan. In theory, based on Watson-Crick base pair formation, if an appropriate target can be identified, an antisense oligomer of more than 15 to 17 nucleotides in length would be expected to have a unique sequence relative to the entire human genome. A suitable oligomer should be able to interfere, in a sequence specific manner with the process of mRNA translation into protein [9]. The requirements for an antisense oligomer for therapeutic use are: (1) that it must be stable in vivo; (2) it must be able to enter the target cell; and (3) it must be able to interact with its cellular targets.

As oligomers possess little or no innate ability to diffuse across cell membranes, the cells must take them up through energy-dependent mechanisms. To resolve the problem of uptake, a large number of strategies have been employed in order to augment the rate of cellular internalization of nucleic acids and to increase the rate at which they pass through the endosomal membrane. These strategies include: (i) coupling oligomers to polycations such as polylysine [10], polyethylamine [11] or others; (ii) use of transferin/polylysine-conjugated DNA in the presence of the capsid of a replication-deficient adenovirus [12]; (iii) conjugation of oligonucleotides to fusogenic peptides [13] or to a peptide fragment of the homeodomain of the Drosophila antennapedia protein [14]; (iv) targeting of oligonucleotides to specific cell surface receptors, such as folate, asialoglycoprotein receptor and transferrin [15], (v) conjugation to cholesterol [16]; and, most successfully (vi) complexation of oligonucleotides with cationic lipids [17] and GS 288 etofectin [18].

Preferred antisense sequences are those designed to comprise sequences which hybridize to uniquely conserved regions in the PAR family of proteins. Conserved regions may be identified by comparing the nucleotide sequences of different members of the PAR family. For example, certain regions within the ThR sequence have 27% sequence similarity to PAR-3 and 28% similarity to PAR-2. Examples of conserved unique regions are:

1) The protease activated domains and hirudin binding domain:

Nucleotides

| | |
|---|---|
| hPAR-1(ThR) | 37-61.....TLDPRSFLLRNPNDKYEPFWEDEEK |
| hPAR-2 | 32-56.......SSKGRSLIGKVDGTSHVTGKGVTVE |
| hPAR-3 | 34-57......TLPIKTFRGAPPN SFEEFPFSALE |
| hPAR-4 | 28-52......LPAPRGYPGQVCANDSDTHELPDSS |

2) Second extracellular loop: located between transmembrane domains 4 & 5 and corresponding to residues: ITTCHDV which are conserved in PAR 1-3, while in PAR-4 only the three amino acids CHD are conserved.
3) The entire promoter region of the PAR family (i.e. 5′ cloned regions downstream to the ATG of PAR-1 and PAR-3). This region is likely to contain important regulatory sequences.

### DETAILED DESCRIPTION OF THE DRAWINGS

In order to understand the invention and to see how it may be carried out in practice, a preferred embodiment will now be described, by way of non-limiting example only, with reference to the accompanying drawings, in which:

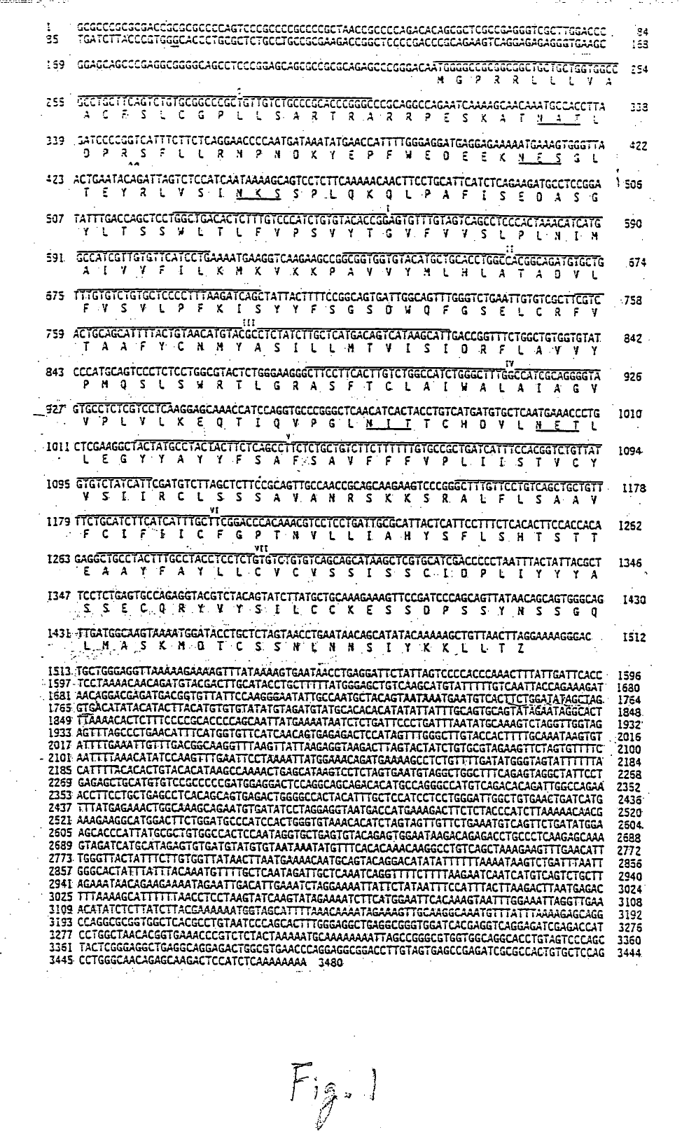

FIG. 1 shows the DNA and amino acid sequence of human ThR [1];

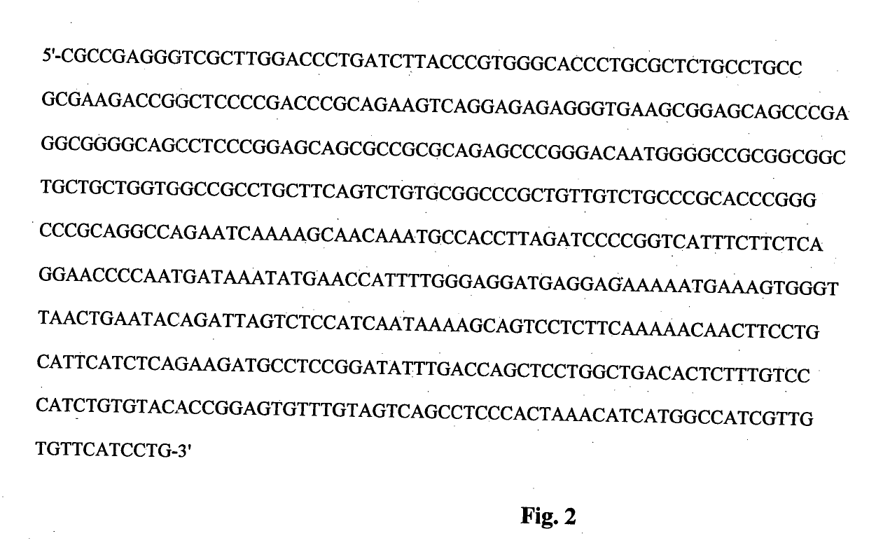

FIG. 2 shows the DNA sequence of an antisense cDNA of ThR;

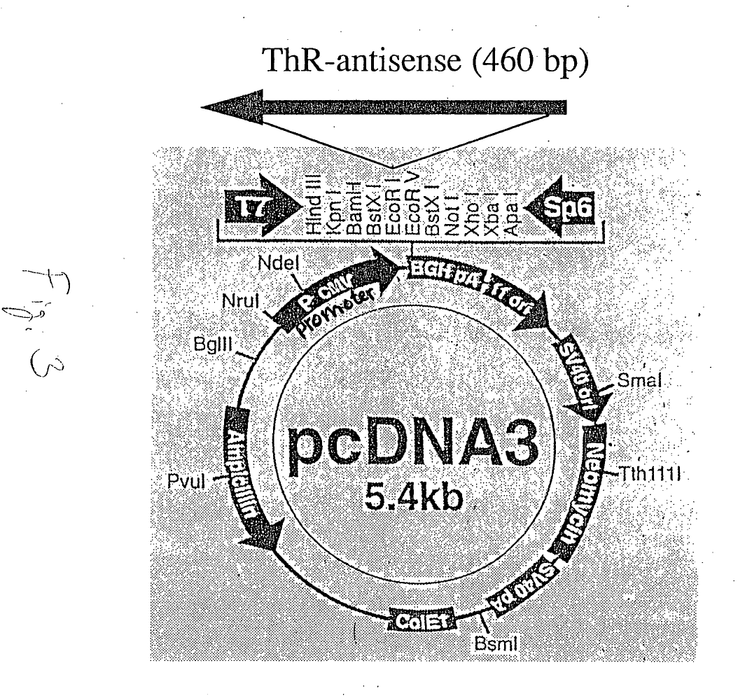

FIG. 3 shows the location of the ThR antisense in the pcDNA III vector;

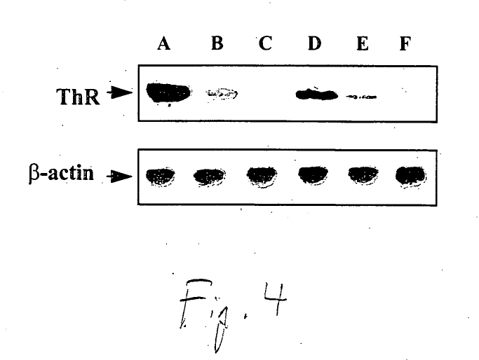

FIG. 4 illustrates ThR expression in human breast carcinoma cell lines. Total RNA isolated from, human breast carcinoma cell-lines was analyzed by Northern blotting. The cell lines used were: MDA-435 (A), MDA-231 (B) and MCF-7 (C), as well as Ha-ras-transfected breast carcinoma cell lines, MCF10AT3B (D), MCF10AT (E) and MCF10A (F). The blots were probed with ³²P-labeled 250 base pair DNA, corresponding to ThR (upper part), or with ³²P-labeled β-actin DNA (lower part).

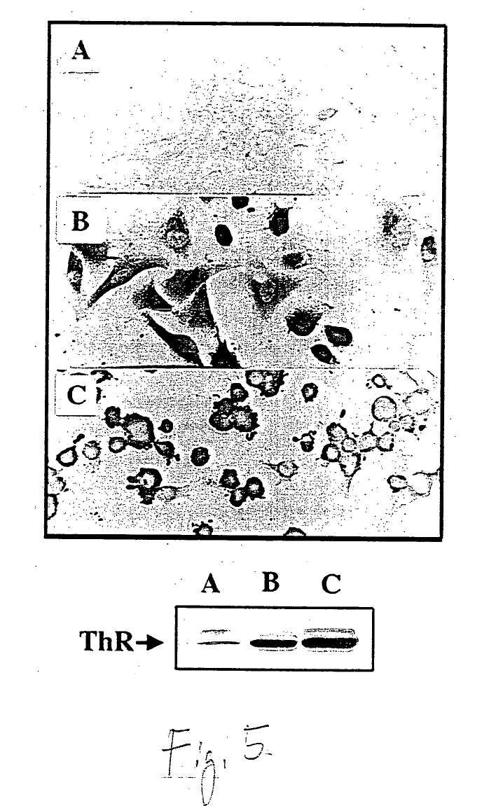

FIG. 5 illustrates immunocytochemical analysis of cell-associated ThR. Human breast carcinoma cell lines (MCF-7, MDA-231, and MDA-435) were cultured in 8-well chamber slides and analyzed for the presence of ThR. Specific staining of the receptor was obtained following incubation with affinity purified polyclonal anti ThR antiserum followed by biotin conjugated goat-anti-rabbit IgG antibodies and detected by extravidin incubation. Photographs of representative areas of MCF-7 (a), MDA-231 (b) and MDA-435 (c) cell monolayers are shown (x400).

Lower Panel. Western blot analysis of ThR. Western blot analysis of cell lysates (50 μg/lane) of MCF-7 (A), MDA-231(B) and MDA-435 (C) cells. Specific protein band was detected following incubation with anti ThR antibodies and visualized by the ECL immunoblotting detection system according to the manufacturer's instructions.

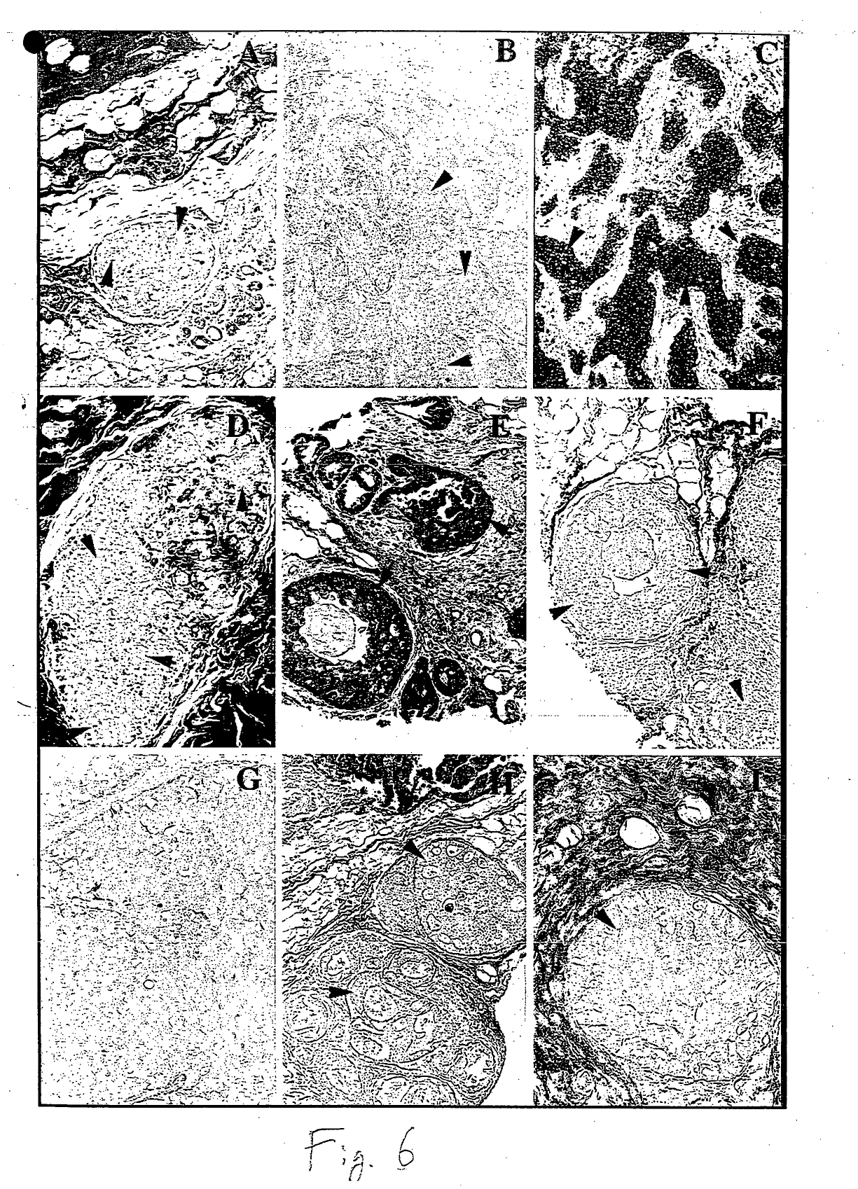

FIG. 6 illustrates in situ hybridization of ThR mRNA in normal and cancerous breast tissue specimens. Hybridization with ThR riboprobes was performed on: Normal breast duct lobular units (A&D). Invasive duct carcinoma, (IDC) (antisense orientation, C; sense orientation, B). High grade DCIS of comedo type (antisense orientation, E, sense orientation, F). Low grade DCIS, solid type (G) and atypical intraductal hyperplasia (AIDH, H &I). Detection of specifically hybridized mRNA to DIG-labeled probe was performed using anti-DIG-alkaline phosphatase conjugated antibodies (Boehringer Mannheim, Mannheim, Germany). These analyses represent at least 3 patients of each category.

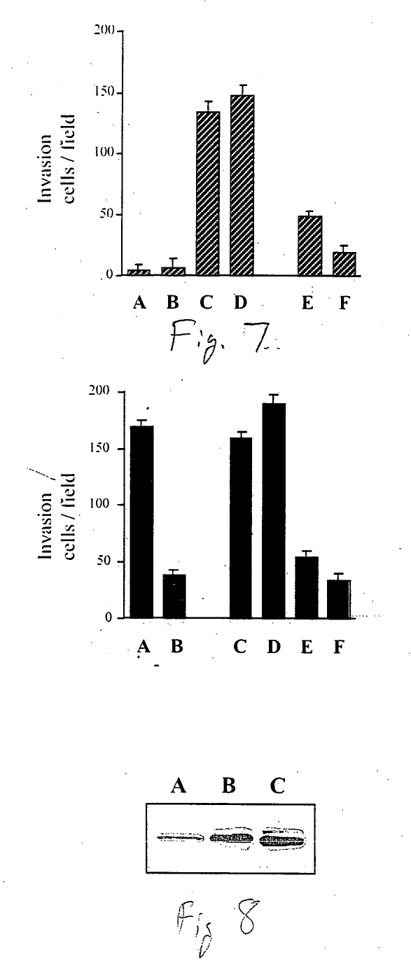

FIG. 7 illustrates Matrigel invasion of breast carcinoma cell lines. The indicated cells (ZR-75, A; MCF-7, B; MDA-435, C; MDA-231, D; fibrocystic MCF10AT3B, E; fibrocystic MCF10A, F) were applied (2×10⁵ cells/assay) to the upper compartment of Boyden chambers. Cell invasion through Matrigel coated filters was determined, as outlined in Materials and Methods, below.

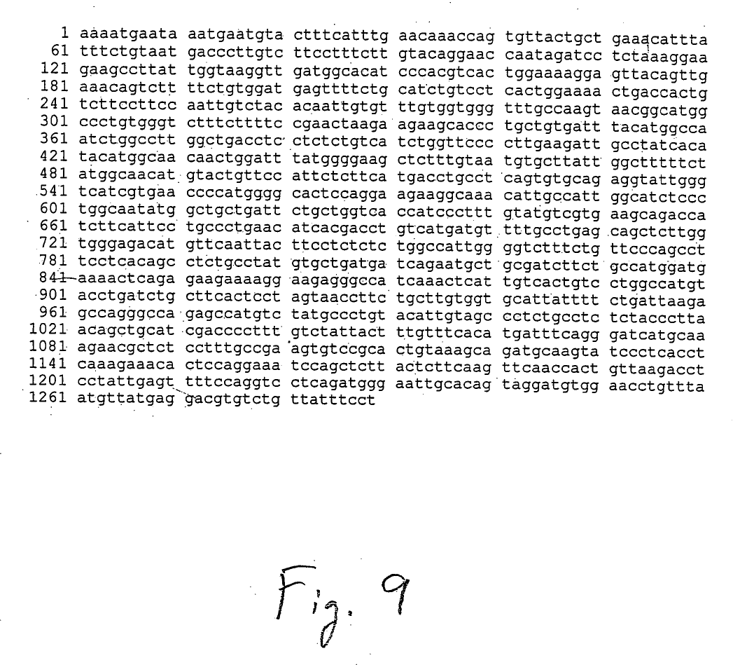

FIG. 8 illustrates inhibition of MDA-435 Matrigel invasion by ThR antisense. MDA-435 cells were transiently transfected with pCDNAIII expression plasmid containing the antisense ThR of FIG. 2. The level of invasion was compared to untreated MDA-435 (A) and MCF-7 (B) cells. Control transfections of MDA-435 cells were performed in the presence of vector alone—(C) or DOTAP liposomes alone (Gibco—BRL) (D). Nearly confluent (60%) cells were treated with various concentrations of the plasmid: transfection with antisense ThR—5 μg/plate (E), transfection with antisense ThR—20 μg/plate (F). The invasion assay was performed as described under Materials and Methods, 72 h following transfection. Lower panel. Western blot analysis of ThR antisense transfectants. MDA-435 cell lysates (50 μg/lane) of ThR antisense transfectants (A) were applied on SDS-PAGE and the level of receptor protein was compared to cells transfected with vector alone (B) or untreated cells (C).

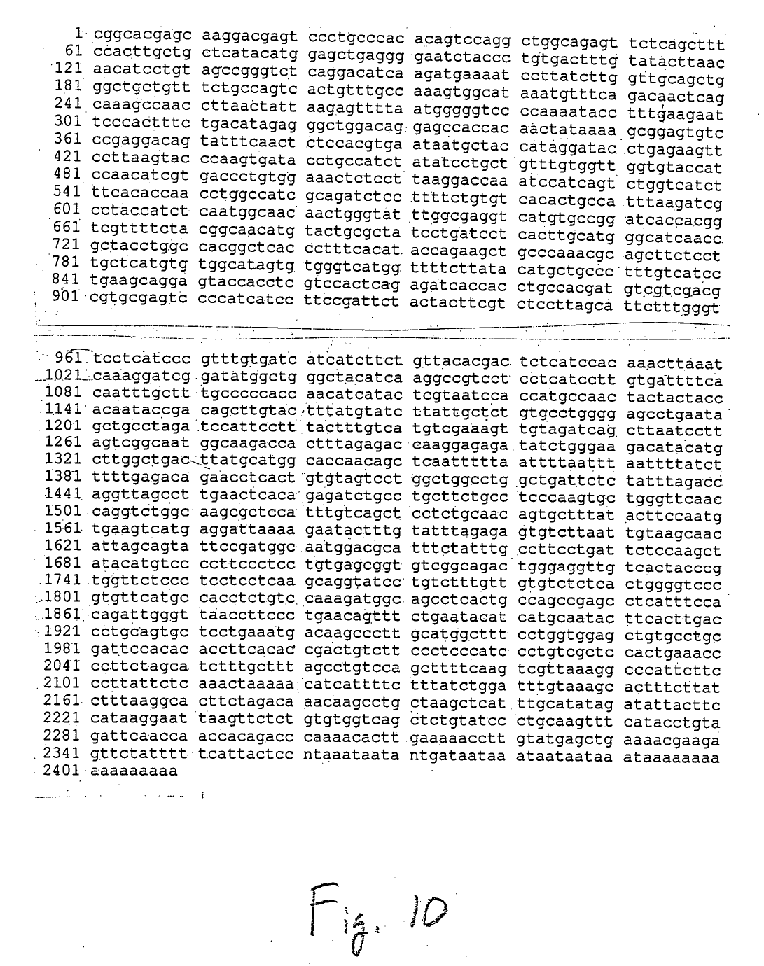

FIG. 9 shows the DNA sequence of PAR-2;

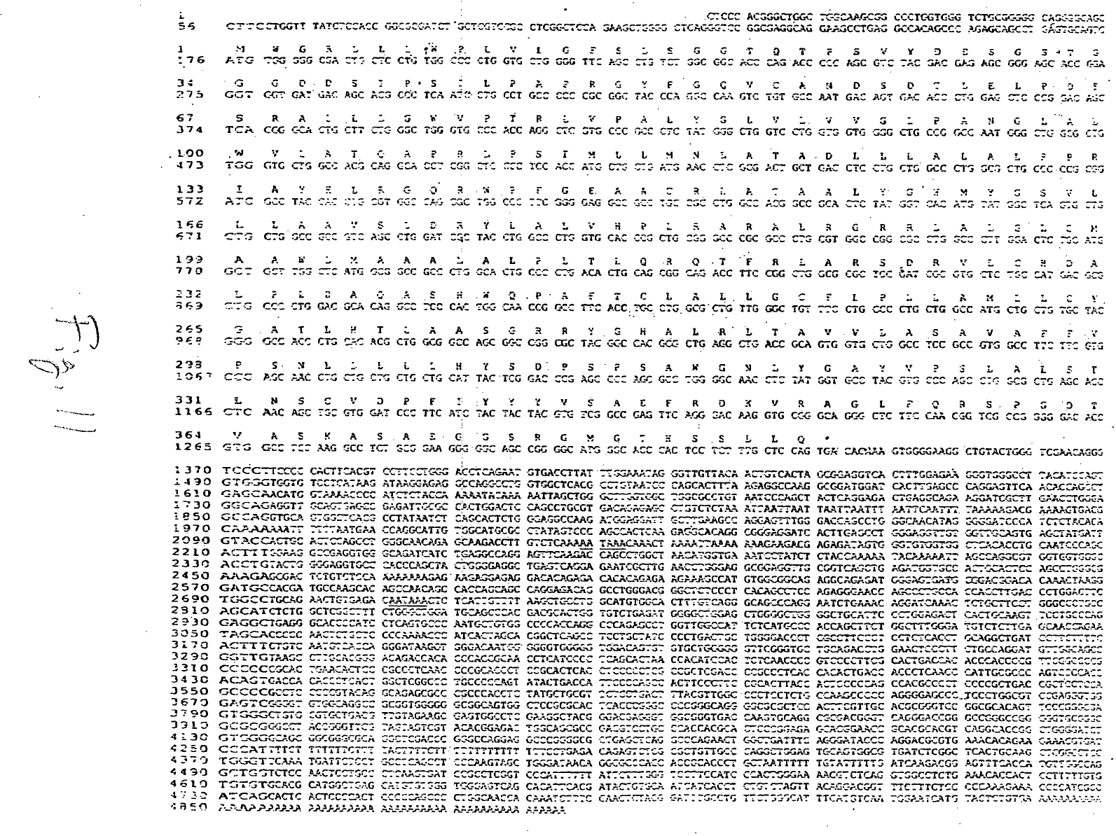

FIG. 10 shows the DNA sequence of PAR-3;

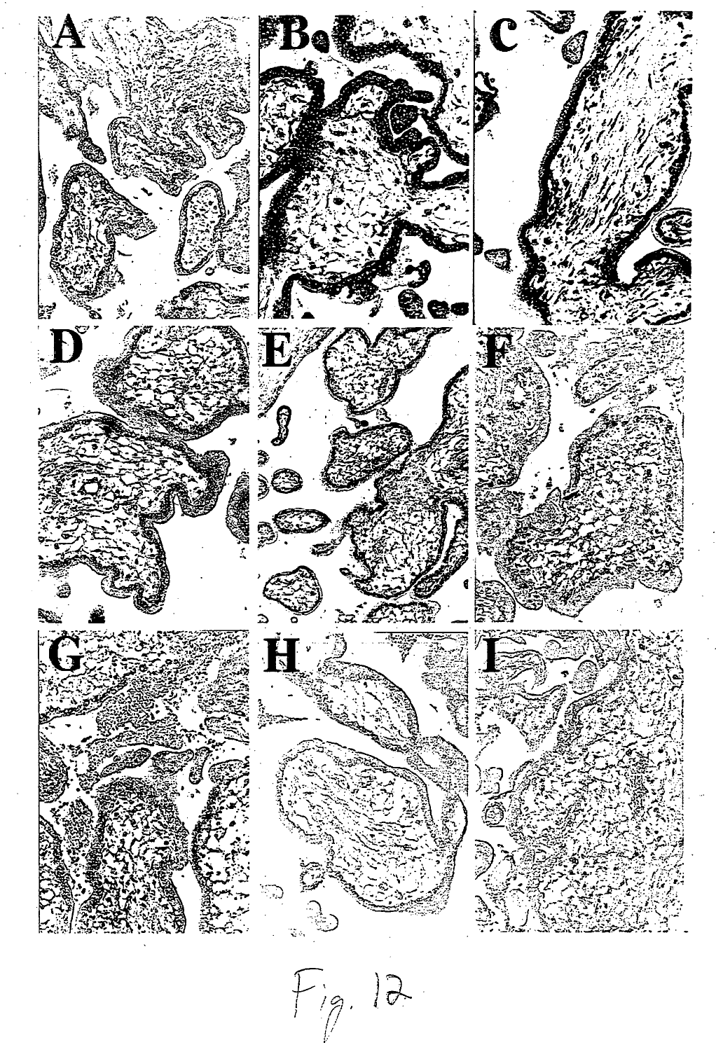

FIG. 11 shows the DNA sequence of PAR-4; and

FIG. 12 illustrates expression of ThR in first trimester human placenta. In situ hybridization analysis of ThR expression at 6-15 weeks of gestation. Placental tissue was obtained from elective termination of pregnancies by dilatation and curettage. Sections of 6 week placental tissue (A) and of 7, 8, 9 and 10 weeks of gestation (B-E, respectively), as visualized by ThR staining of cytotrophoblasts. No staining was observed at weeks 11 and 15 (F & G, respectively). Control hybridization (weeks 7 and 8) using sense orientation showed background staining (H & I, respectively).

### DETAILED DESCRIPTION OF A PREFERRED EMBODIMENT

Materials and Methods

Cells: The human breast carcinoma cell lines, MCF-7 (adenocarcinoma), MDA-MB-231 (adenocarcinoma), MDA-MB-435 (ductal carcinoma) and ZR-75-1 (carcinoma), were kindly provided by Dr. Robert Stern (Department of Pathology, University of California, San Francisco). The invasive properties of these breast cell lines were determined following injection of the cells into the mammary pads of nude mice with or without Matrigel [5]. Cells were cultured in DMEM (1 g glucose/liter) containing 10% bovine calf serum. MCF10A (nearly-normal immortalized epithelial cells), MCF10AT cells (derived from human fibrocystic epithelium transfected with Ha-ras) and MCF10AT3B cells (derived from a 94-day third transplant generation of lesion in Beige/Nude mice, classified as grade 2), were kindly provided by Dr. F. R. Miller (Karamanos Cancer Institute, Meyer L. Prentiss Center, Detroit) and grown in RPMI-1640 containing 10% fetal calf serum (FCS). Tissue culture medium was supplemented with penicillin (50 U/ml) and streptomycin (50 μg/ml) (GIBCO-BRL, Gaithersburg, Md.) and the cells were maintained at 37° C. in a 10% CO₂ humidified incubator. Cells were dissociated with 0.05% trypsin/0.02% EDTA, 0.01M sodium phosphate (pH 7.4) solution (STV) and subcultured at a split ratio of 1:5.

Plasmids and transfection: The DNA and amino acid sequences of ThR are shown in FIG. 1 [1]. ThR in the antisense orientation (FIG. 2), consisting of 612 nucleotides (from (−)75 to (+)537 of FIG. 1) was prepared and inserted into the eukaryotic expression plasmid, pcDNA II (Invitrogene, Carlsbad, Calif.) at the HindIII and EcoRI sites (FIG. 3). Antisense ThR cDNA was used for transient transfection experiments. Subconfluent (25-40%) MDA-435 breast cancer cells were grown in 60 mm culture dishes and a total of 5-20 μg of DNA and DOTAP—transfection reagent (10 μg DOTAP/μg DNA; 4.5 h incubation, Boehringer Mannheim, Mannheim, Germany) were used for transfection. Cells were assayed 48-72 h following transfection.

RNA Isolation and Northern blot analysis: RNA was prepared using TRI-Reagent (Molecular Research Center, Inc. Cincinnati) according to manufacturer's instructions. The RNA (20 μg of total RNA) was separated by electrophoresis through a 1.1% agarose gel containing 2 M formaldehyde, transferred to a nylon membrane (Hybond N⁺; Amersham) and hybridized either to cDNA probes or PCR product radiolabeled by random primer extension with [α-³²P]Dct [6] for 24 h at 42° C. The membrane was washed twice for 30 min at room temperature with 2×SSC containing 2% SDS and 15 min at 50° C. with 0.1×SSC, containing 0.1% SDS. The blots were exposed for 2-4 d at −70° C. and the relative amounts of mRNA transcripts were analyzed by laser densitometry using an Ultroscan XL Enhanced Laser Densitometer and normalized relative to internal β-actin controls.

In situ hybridization of human tumor and placenta biopsy specimens. RNA probes were transcribed and labeled by T₇ RNA polymerase (for antisense orientation) or T₃ RNA polymerase (for sense control orientation) using DIG-UTP labeling mix (Boehringer Mannheim, Mannheim, Germany). Probes were labeled from plasmid containing 462 base pair fragments of the human ThR (pBhThR-462S) inserted into the EcoRI-HindIII site. Final concentration for hybridization was 1 μg/ml, according to the manufacturer's instructions for non radioactive in situ hybridization application. Hybridization was carried out (overnight, 45° C.) on paraffin embedded breast tissue sections (Department of Pathology, Hadassah University Hospital, Jerusalem) or placenta sequential sections. Slides were washed in 0.2×SSPE (3×1 h) at 50° C. and blocked by blocking reagent (Boehringer Mannheim, Mannheim, Germany). Detection was performed using AP-conjugated, anti-DIG antibodies (Fab-fragment, diluted 1:300; Boehringer Mannheim, Mannheim, Germany), overnight at room temperature. AP reaction was detected by NBT/BCIP reagents according to the manufacturer's instructions.

Immunohistochemistry: Tumor cells were cultured overnight at 37° C. on eight chamber slides. The cells were fixed with 2% formaldehyde and 2% sucrose/PBS at room temperature for 30 min and permeabilized with 20 mM Hepes, pH 7.4, 300 mM sucrose, 50 mM NaCl, 3 mM MgCl₂ and 0.5% Triton X-100, for 4 min at 0° C. After rehydration with PBS, the cells were incubated (10 min, 24° C.) with 3% H₂O₂ in PBS containing 10 mM glycine, 10 mg/ml BSA, followed by 30 min blocking with normal goat serum in PBS containing 1% BSA. Affinity purified rabbit-anti-human ThR antibodies were added (dilution 1:50-1:200) for 4 h at 4° C., followed by incubation (1 h, room temperature) with a second antibody goat-anti-rabbit IgG-Biotin conjugated and 1 h incubation with HRP-ExtraAvidin (1:200) (Sigma Immuno Chemicals, St. Louis, Mo.).

Antibodies: We have raised anti-ThR antibodies directed toward a synthetic peptide (thrombin-receptor activating peptide; TRAP) corresponding to residues Ser42-Lys51 (i.e. S-F-L-L-R-N-P-N-D-K). KLH conjugated peptide was injected to rabbits, and the immune serum was affinity purified. ELISA was performed on plates coated with the TRAP-peptide showing efficient positive identification at 1:25,600 dilution. Maximal response was obtained at 1:3,200 dilution. Monoclonal anti ThR Abs (mouse IgG1 clone IIaR-A) were used for Western blot analysis (Biodesign, Me., USA)

Western blotting analysis: Cells were dissolved in lysis buffer containing 10 mM Tris-HCl, pH 7.4, 150 mM NaCl, 1 mM EDTA, 1% Triton X-100 and protease inhibitors (5 μg/ml aprotinin, 1 μM phenylmethylsulfonylfluoride and 10 μg/ml leupeptin) for 30 min at 4° C. After centrifugation at 10,000 g for 20 min at 4° C., the supernatants were transferred and the protein content was measured. Lysates (50 μg) were loaded and resolved on 10% SDS-PAGE followed by transfer to Immobilon-P membrane (Millipore, Mass.). Membranes were blocked and probed with anti-ThR antibodies (1:4000) in 1% BSA in 10 mM Tris-HCl (pH 7.5), 100 mM NaCl and 0.05% Tween-20). After washes, blots were incubated with the appropriate second antibodies and conjugated to horseradish peroxidase. Immunoreactive bands were detected by the enhanced chemiluminescence (ECL) reagent using luminol and p-cumaric acid (Sigma, St. Louis, Mo).

Placental tissue sections: Sections of placental tissue, 6-15 weeks of gestation, were obtained from elective termination of normal pregnancies by dilatation and curettage.

Matrigel invasion assay: Blind well chemotaxis chambers with 13 mm diameter filters were used for this assay. Polyvinylpyrrolidone-free polycarbonate filters, 8 μm pore size (Costar Scientific Co., Cambridge, Mass.), were coated with basement membrane Matrigel (25 μg/filter) as previously described [7]. Briefly, the Matrigel was diluted to the desired final concentration with cold, distilled water, applied to the filters, dried under a hood, and reconstituted with serum-free medium. Cells (2-3×10⁵), suspended in DMEM containing 0.1% bovine serum albumin were added to the upper chamber. Conditioned medium of 3T3 fibroblasts was applied as a chemoattractant and placed in the lower compartment of the Boyden chamber. Assays were carried out at 37° C. in 5% CO₂; Over 90% of the cells attached to the filter after a 2 h incubation. At the end of the incubation, the cells on the upper surface of the filter were removed by wiping with a cotton swab. The filters were fixed in methanol and stained with hematoxylin and eosin. Cells in various areas of the lower surface were counted and each assay was performed in triplicate. For chemotaxis studies, filters were coated with collagen type IV alone (5 μg/filter) to promote cell adhesion. Cells were added to the upper chamber and conditioned medium was applied to the lower compartment.

### EXAMPLES

### Example I

### ThR Expression in Breast Carcinoma Cell Lines

In a preliminary experiment, a panel of mammary carcinoma cells was surveyed for a possible correlation between the level of ThR expression and established degrees of metastasis (FIG. 4). The cell lines used included one near-normal diploid immortalized breast epithelial cell line (MCF10A) originating from fibrocystic disease, and 6 tumor cell lines exhibiting different doubling times, tumorigenicity and metastases in nude mice. Of these cell lines, MDA-435 (a highly metastatic cell line), and MCF10AT3B (ras transfected fibrocystic epithelium reestablished several times from lesions formed in nude mice), were compared to medium metastatic (MDA-231 and MCF10AT, ras transfected fibrocystic cells), or carcinoma cells exhibiting no metastatic potential (ZR-75 and MCF-7 cells). As shown in FIG. 4, high levels of ThR mRNA were found in the highly aggressive cells (lanes A, D) as compared to moderate levels in MDA-231 and ras transfected fibrocystic cells (lanes B& E, respectively), and no expression in the non-metastatic MCF-7 and MCF10AT cells (lanes C&F, respectively). The mRNA levels were quantified by densitometric analysis and the ratio of ThR/β-actin in each lane was calculated. The ThR mRNA level in MDA-435 was 6 fold higher than in MDA-231 cells (FIG. 4, lanes A vs B) and, as mentioned above, no detectable ThR was observed in MCF-7 cells (FIG. 4, lane C). A similar correlation between ThR level of expression and metastasis was obtained in Ha-ras transfected cells showing a 4 fold higher level in MCF10AT3B (obtained following ras-transfection and xenografting 3 times in mice) than in MCF10AT-ras transfected cells FIG. 4, lanes D vs E). No detectable level of expression was observed in the fibrocystic, non-malignant, epithelial cells, MCF10A epithelial cells (FIG. 4, lane F).

Affinity purified rabbit-anti-human ThR antibodies were applied to detect the expression and localization of the receptor protein. Massive staining of MDA-231 and MDA-435 cells was observed (FIGS. 5B&C, respectively), as opposed to little or no staining of MCF-7 cells (FIG. 5A). In parallel, Western blot analysis showed a distinct protein band of ThR in MDA-435 cells (FIG. 5, lower panel; lane C), somewhat reduced ThR level in MDA-231 (lower panel; lane B) and little or no protein in MCF-7 breast carcinoma cells (lower panel; lane A).

Collectively, these data demonstrate the preferential expression of ThR in metastatic breast carcinoma cell lines, but not in non-metastatic MCF-7 or MCF10A breast carcinoma cells, regardless of whether the mRNA or protein levels were evaluated.

### Example 2

### ThR Expression in Human Breast Tissue Specimens

ThR gene expression and localization in vivo was studied in formalin fixed paraffin embedded human breast carcinoma specimens as compared to normal mammary sections obtained from reduction mammoplasty. ThR expression was examined in primary breast tumors representing poor to benign prognosis. In situ hybridization analysis using a ThR RNA probe (corresponding to nucleotide nos. 320-570 of the sequence of FIG. 1) was performed with an archival set of paraffin embedded biopsy specimens. A total of 10 normal-breast tissue specimens, and 8 specimens of infiltrating ductal carcinoma were analyzed. The invasive carcinoma specimens were selected from typical infiltrating duct carcinoma of high nuclear grade with numerous atypical mitotic figures and with evidence of vascular invasion and lymph node metastases.

As demonstrated in FIG. 6, hybridization of a ThR antisense RNA probe to invasive duct carcinoma specimens resulted in strong positive staining localized specifically to the carcinoma cells (FIG. 6C). Weaker positive staining was noted in high-grade ductal carcinoma in situ (DCIS) of comedo-type (FIGS. 6 E&F). In contrast, very little or no staining was observed in low-grade, solid type DCIS (FIG. 6G), and no staining was observed in premalignant atypical intraductal hyperplasia (AIDH) (FIGS. 6 H&I) and in normal breast duct lobular units (FIGS. 6 A&D; note that the high staining seen in the background is limited to the fibers, and is not seen in the epithelial cells). AIDH was distinguished from low grade DCIS, non-comedo type according to the diagnostic criteria of Dupont, Page and Rogers [8]. Expression was also noted in some cases of DCIS, in particular, high grade, comedo-type lesions. The low grade DCIS of solid type showed weak to no expression of ThR, while cases of AIDH, as well as normal breast tissue from reduction mammoplasty specimens did not show any expression of ThR.

### Example 3

### Antisense ThR Inhibits Metastatic Breast Carcinoma Cell Invasion

To assess the invasion properties of aggressively metastatic breast carcinoma cells, the Matrigel in vitro invasion assay was applied. For this purpose, a reconstituted matrix of basement membrane was utilized to coat porous filters, in order to closely mimic natural barriers in a Boyden chamber. As a chemoattractant source, fibroblast conditioned medium was placed in the lower compartment [7]. The Matrigel invasion assay confirmed the expected differential metastatic properties of the carcinoma cell lines. High levels of invasion through Matrigel were obtained with MDA-435 and MDA-231 cells (FIGS. 7, D&C). MCF10AT3B-ras transfected fibrocystic cells invaded the Matrigel to a lower extent (FIG. 7, E), while no movement was detected with the MCF10AT, MCF-7, or ZR-75 non-metastatic cell lines (FIGS. 7, F & A,B, respectively).

To analyze the impact of reduced ThR expression in the highly metastatic cells, MDA-435 breast carcinoma cells were transfected with an antisense ThR cDNA. mammalian expression vector containing ThR cDNA in an antisense orientation under the control of the Cytomegalovirus (CMV) promoter (see FIGS. 2 and 3). The vector alone was used as a control. Western blot analysis of ThR protein levels showed a marked reduction in the antisense transfected cells (FIG. 8, lane A) as compared to vector alone (lane B) or untreated MDA-435 cells (lane C). When the antisense transfected cells were tested in the Matrigel invasion assay, the otherwise aggressively invading cells showed a markedly reduced level of invasion, similar to that of the non-metastatic breast carcinoma cell line MCF-7 (FIGS. 8, E&F). Transfection with the vector alone had no effect on the invasion properties and the transfected cells migrated effectively through the Matrigel layer (D), similar to the metastatic MDA-435 cells (A).

Similar antisense molecules may be prepared from other members of the PAR family, such as PAR-2 (FIG. 9), PAR-3 (FIG. 10) and PAR-4 (FIG. 11).

### Example 4

### ThR Expression During Placenta Development

Human embryo development depends on proper placentation and successful implantation. Trophoblast invasion through the uterine epithelium and deep into the stroma enables the establishment of the proper fetal-maternal interactions. Histological examination of placental biopsies during the first trimester (6-15 weeks), obtained from elective termination of pregnancies, showed a striking pattern of ThR temporal regulation. ThR mRNA levels were not detected up to 6 weeks of gestation (FIG. 12,A), increased markedly between 7-10 weeks (B-E), then fell precipitously at 11 weeks and thereafter (F&G). The staining was specific to ThR, since hybridization with ThR sense orientation on placental biopsies taken on weeks 7 and 8, showed no staining (H&I, respectively). The receptor appeared localized to the cytotrophoblasts within the vim, and also, to some extent, in the syncytiotrophoblasts of the invading column.

### REFERENCES

1. Vu, T-K., Hung, H. D. T., Wheaton V. I. & Coughlin, S. R. Molecular cloning of a functional thrombin receptor reveals a novel proteolytic mechanism of receptor activation. Cell 64, 1057-1068 (1991).
2. Nystedt, S., Emilsson, K., Wahlestedt, C. & Sundelin, J. Molecular cloning of a potential proteinase activated receptor. Proc. Natl. Acad Sci. USA 91, 9208-9212 (1994).
3. Nystedt, S., Emilsson, K., Larsson, A-K., Strombeck, B. & Sundelin, J. Molecular cloning and functional expression of the gene coding for the human proteinase-activated receptor 2. Eur. J Biochem 232, 84-89 (1995).
4. Ishihara, H. et al. Protease-activated receptor-3 is a second thrombin receptor in humans. Nature 386, 502-506 (1997).
5. Gincotti, F. G. & Ruoslahti, E. Elevated levels of the α₅β₁ fibronectin receptor supresses transformed phenotype of Chinese hamster ovary cells. Cell 60, 849-859 (1990).
6. Feinberg, V. & Vogelstein, B. A technique for radiolabeling DNA restriction endonuclease fragments to high specific activity. Anal. Biochem 132, 6-13 (1984).
7. Albini, A. et al. A rapid in vitro assay for quantitating the invasive potential of tumor cells. Cancer Res. 47, 3239-3245 (1987).
8. Page, D. L. et al. Atypical hyperplastic lesions of the female breast: a long term follow-up study. Cancer 55, 2698-2708 (1985).
9. Toulme, J. and Helene, C. (1988) Gene 72, 51.
10. Clarene. J. P., Degols, G., Leonetti, J. P., Milhaud, P. & Lebleu, B. (1993) Anticancer Drug Design, 8, 81.
11. Boussif, O., Lezoulac'h, F., Zanta, M. A., Mergny, M. D., Scherman, E., Demeneix, B. & Behr (1995) Proc. Natl. Acad. Sci. USA 92, 7297.
12. Zatloukal, K., Wagner, E. & Cotton, M. (1992) Proc. Natl. Acad Sci. USA 600, 136.
13. Bongartz, J. P., Aubertin, A. M., Milhaud, P. G. & Lebleu B.,
(1994) Nucleic Acids Res. 22, 4681.
14. Derossi D., Jolit, A. H., Chassaing, G.& Prochiantz, A., (1994) J. Biol. Chem. 269, 10444.
15. Citro, G., Perrotti, D., Cucco, C., D'Agnano, I., Sacchi, A., Zupi G. & Calabretta, B. (1992) Proc. Natl. Acad Sci. USA 89, 7031.
16. Ink, N. H., Beekman, J. M., Kessler, D. J. Murphy, M., Jayaraman, K., Zendegui, J. G., Hogan, M. F., O'Malley, B. W. & Tsai, M. J. (1993) Nucleic Acids Res. 21. 2789.
17. Bennett, C. F., Chiang, M. Y., Chan, H., Shoemaker, J. E. & Mirabelli, C. K. (1992) Mol. Pharmacol. 41, 1023.
18. Lewis, J. G., et al. (1996) Proc. Natl. Acad Sci. USA 93, 3177.
19. Xu, et al. (1998) Proc. Natl. Acad Sci. USA 95, 6642-6646.

1. A method for treating metastatic tumor cells of a subject comprising administrating to said subject an antisense molecule, said antisense molecule comprising a nucleotide sequence which is complementary to an RNA sequence of a protease activated receptor (PAR) protein.

2. A method according to claim 1 wherein said PAR protein is a thrombin receptor.

3. A method according, to claim 1 wherein said PAR protein is selected from the group consisting of PAR-2, PAR-3 and PAR-4.

4. A method according to any of claims 1-3 wherein said tumor cell is of epithelial tissue origin.

5. A method according to claim 4 wherein said epithelial tissue is selected from the group consisting of breast, esophagus, kidney, prostate, ovary, melanoma and bladder.

6. A method according to any of claims 1-5 wherein said antisense molecule has the sequence appearing in FIG. 2.

7. A method for treating metastatic tumor cells of a subject comprising administrating to said subject an antibody molecule, said antibody molecule being capable of binding to a protease activated receptor (PAR) protein.

8. A method according to claim 9 wherein said antibody binds an extracellular epitope of said PAR protein.

9. An antisense molecule comprising a nucleotide sequent which is complementary to an RNA sequence of a protease activated receptor (PAR) protein.

10. A pharmaceutical composition comprising an active factor and a pharmaceutically acceptable carrier, said active factor being an antisense molecule comprising a nucleotide sequence which is complementary to an RNA sequence of a protease activated receptor (PAR) protein.

11. A pharmaceutical composition according to claim 10 for the treatment of metastatic tumor cells.

12. A pharmaceutical composition according to claim 11 wherein said PAR protein is a thrombin receptor.

13. A pharmaceutical composition according to claim 11 wherein said PAR protein is selected from the group consisting of PAR-2, PAR-3 and PAR-4.

14. A pharmaceutical composition according to any of claims 11-13 wherein said tumor cell is of epithelial tissue origin.

15. A pharmaceutical composition according to claim 14 wherein said epithelial tissue is selected from the group consisting of breast, esophagus, kidney, prostate, ovary, melanoma and bladder.

16. A pharmaceutical composition according to any of claims 11-15 wherein said antisense molecule has the sequence appearing in FIG. 2.

17. A method for the treatment of disorders involving the implantation of a placenta in a female subject comprising administrating to said subject an antisense molecule, said antisense molecule comprising a nucleotide sequence which is complementary to an RNA sequence of a protease activated receptor (PAR) protein.

18. A method according to claim 13 wherein said antisense molecule is administered to a trophoblast cell.

19. A pharmaceutical composition according to claim 10 for the treatment of disorders involving the implantation of a placenta in a female subject.
